(19) United States
(12) Patent Application Publication
(10) Pub. No.: US 2011/0118623 A1
(43) Pub. Date: May 19, 2011

(54) ELECTRONIC THERMOMETER

(75) Inventors: Yoshihito NAKANISHI, Suita-shi (JP); Katsuyoshi MORITA, Nagaokakyo-shi (JP); Takeshi HAGIMOTO, Ikoma-shi (JP)

(73) Assignee: OMRON HEALTHCARE CO., LTD., Kyoto-shi (JP)

(21) Appl. No.: 13/008,467

(22) Filed: Jan. 18, 2011

Related U.S. Application Data
(63) Continuation of application No. PCT/JP2009/063181, filed on Jul. 23, 2009.

(30) Foreign Application Priority Data
Jul. 28, 2008 (JP) 2008-193952

(51) Int. Cl. A61B 5/01

(52) U.S. Cl. 600/549

(57) ABSTRACT

The invention provides an electronic thermometer in which a contact state with a human body can be confirmed by a simple, easy-to-assemble configuration. The electronic thermometer includes a hollow outer case that includes a probe unit with a temperature measuring unit, a temperature sensor, an inner case, a control circuit, and a pair of electrodes. The electrodes are positioned inside the probe unit by mounting the inner case on the outer case. A determination unit is also provided and the determination unit measures an electrostatic capacitance between the pair of electrodes and determines whether the probe unit is in proper contact with the measured region of the user based on a change of the measured electrostatic capacitance.

### TECHNICAL FIELD

The present invention relates to an electronic thermometer.

### BACKGROUND ART

Conventionally, there is well known an electronic thermometer that can correctly measure a body temperature by sensing whether a human body is in contact with a temperature measuring unit in which a temperature sensor is disposed.

As to this kind of electronic thermometer, for example, Patent Document 1 describes an electronic thermometer in which a switch, a contact resistance, an electrostatic capacitance, humidity, pressure (contact point), temperature comparison, a change in temperature, and the like are utilized as a method for sensing the contact with the human body.

However, in order to correctly sense the contact state with the human body, it is necessary that a sensing unit be assembled while disposed at a proper position. There is also a problem in assembly because a component configuration becomes complicated compared with a usual electronic thermometer that does not include the sensing unit. Particularly, in the configuration described in Patent Document 1, a contact sensing unit is provided while exposed to a surface of a temperature taking probe, the contact sensing unit assembling work involving internal wiring becomes the work on fitting the contact sensing unit in a hole made in part of the temperature taking probe, thereby degrading workability. Accordingly, it is considered that the temperature taking probe is divided to assemble the contact sensing unit such that the contact sensing unit is covered with the temperature taking probe. However, it is necessary to fix the portion in which the temperature taking probe is divided, which increases the number of working processes.

When a proper contact sensing position varies according to a body type of a user, it is considered that multiple temperature taking probes having different positions at which the sensing unit is placed are used. However, in such cases, it is necessary to prepare the temperature taking probe and the contact sensing unit, which are suitable to the body types of the users, which causes a problem in productivity.

Patent Document 2 proposes an electronic thermometer in which the contact sensing unit is formed by a conductive paste. However, it is necessary that the conductive paste is integrated with a sheet, which complicates the configuration of the electronic thermometer.

### PRIOR ART DOCUMENTS

### Patent Documents

Patent Document 1: Japanese Unexamined Patent Publication No.
Patent Document 2: Japanese Unexamined Patent Publication No. 2007-195618

### DISCLOSURE OF THE INVENTION

### Problems to be Solved by the Invention

The present invention is made to solve the above problems of the related art, and an object of the invention is to provide an electronic thermometer in which the contact state with the human body can be confirmed by the simple, easy-to-assemble configuration.

### Means for Solving the Problem

In order to achieve the object of the invention, an electronic thermometer according to an aspect of the invention includes: a hollow outer case that includes a probe unit, the probe unit including a temperature measuring unit that abuts on a measured region of a user at a leading end thereof, a temperature sensor being disposed in the temperature measuring unit in order to detect a temperature; an inner case that is mounted on a hollow center of the outer case while a electronic circuit board is attached to the inner case, a control circuit that processes data detected with the temperature sensor being formed in the electronic circuit board; and a pair of electrodes that is fixed to the inner case, the electrodes being positioned inside the probe unit by mounting the inner case on the outer case, wherein a determination unit is provided in the control circuit, the determination unit measuring an electrostatic capacitance between the pair of electrodes and determining whether the probe unit is in proper contact with the measured region of the user based on a change of the measured electrostatic capacitance.

The electrostatic capacitance between the pair of electrodes disposed in the hollow center of the probe changes when the probe unit comes into contact with the measured region by sandwiching the probe in the underarm of the user. The determination whether the probe unit is in proper contact with the measured region of the user can be made based on the change in electrostatic capacitance.

According to the configuration, the electrode that detects the contact state with the human body is disposed in the hollow center of the outer case, so that the outer case identical to conventional one can be used. That is, it is not necessary to change the shape of the outer case to a special shape in which the electrode can be disposed. The electrode can be positioned at a proper detection point inside the probe unit by mounting the inner case on the hollow center of the outer case, which facilitates the electrode attaching work.

Examples of the case where the probe unit comes into contact with the measured region of the user includes the case where the whole probe unit is tightly sandwiched in the underarm while the leading end of the probe unit at which the temperature sensor disposed abut firmly on the deepest portion of the underarm and the case where the whole probe unit is firmly held between a tongue and a lower jaw while the leading end of the probe unit abuts firmly on the sublingual region.

The pair of electrodes may be disposed in a longitudinal direction of the probe unit while separated from each other with an interval.

Therefore, a gap is formed between end faces that are opposite each other in the pair of electrodes in the longitudinal direction of the probe unit. The change in electrostatic capacitance between the electrodes increases as the point with which the human body is in contact comes close to the gap. Therefore, the electrostatic capacitance becomes the maximum when the human body comes into contact with the probe unit so as to circumferentially surround the outer surface of the probe unit along the gap. When the probe unit is sandwiched in the underarm, usually the human body comes into contact with a whole circumference of the outer surface of the probe unit. Accordingly, at this point, the electrostatic capacitance is set to an electrostatic capacitance in the state in which the temperature measuring unit comes into proper contact with the measured region, thereby being able to make the determination whether the temperature measuring unit at the leading end of the probe is firmly sandwiched in the underarm or the like.

The pair of electrodes may be fixed to the inner case by fitting a recess or a projection, provided in the pair of electrodes, and a recess or a projection, provided in the inner case, in each other.

The electrodes are fixed to the inner case by the fitting between the recess and the projection, while allows the electrodes to be correctly positioned while the electrode attaching work is facilitated.

The pair of electrodes and/or the inner case may include the multiple recesses or projections.

Therefore, the dispositions of the electrodes can easily be changed by changing the recess and projection, which are fitted in each other. Accordingly, when the proper detection position varies according to the body type of the user, a product specification can easily be changed by changing the positions at which the electrodes are positioned. That is, it is not necessary to prepare the multiple kinds of inner cases having different positions at which the electrodes are positioned in order to change the product specification, and excellent productivity can be obtained.

Screw fitting units, which are able to be fitted in each other, are provided in the pair of electrodes and the inner case.

The electrodes are fixed to the inner case by fitting the screw fitting units, which facilitates the electrode attaching work. The dispositions of the electrodes can easily and finely be changed by changing the fitting positions. Accordingly, when the proper detection position varies according to the body type of the user, the product specification can easily be changed by changing the positions at which the electrodes are positioned. That is, it is not necessary to prepare the multiple kinds of inner cases having the different positions at which the electrodes are positioned in order to change the product specification, and thus the excellent productivity can be obtained.

An electrode fixing unit in the inner case may include an elastic portion, and the pair of electrodes may be positioned by pressing the electrodes against an inner wall surface of the probe unit such that the electrodes are attached firmly to the inner wall surface of the probe unit.

The change in electrostatic capacitance between the electrodes increases with decreasing distance from the place with which the human body comes into contact to the gap between the electrodes. The electrode is firmly attached to the inner wall surface of the probe unit and, for example, an air layer except the probe unit is not interposed between the electrodes and the human body. Therefore, the detection accuracy can be improved.

The configurations can be adopted while combined as much as possible.

### Effect of the Invention

As described above, in the present invention, the contact state with the human body can be confirmed by the simple, easy-to-assemble configuration.

### BRIEF DESCRIPTION OF THE DRAWINGS

FIG. 1 is a perspective view of an electronic thermometer according to a first embodiment of the invention.

FIG. 2 is an enlarged perspective view illustrating a periphery of a probe unit of the electronic thermometer of the first embodiment of the invention.

FIG. 3 is an enlarged perspective view illustrating part of an inner case of the first embodiment of the invention.

FIG. 4 is a perspective view illustrating the other side of FIG. 3.

FIG. 5 is a perspective view of an electrode of the first embodiment of the invention.

FIG. 6 is a plan view explaining a wiring configuration of the electronic thermometer of the first embodiment of the invention.

FIG. 7 is a sectional view explaining the wiring configuration of the electronic thermometer of the first embodiment of the invention.

FIG. 8 is a graph illustrating a state of a change in electrostatic capacitance when a measured region comes into proper contact with a temperature measuring unit.

FIG. 9 is a schematic block diagram illustrating an electric configuration of the electronic thermometer.

FIG. 10A is a view explaining a principle in which the electrostatic capacitance changes between conductors, and illustrates a state of a charge between electrodes when a human body does not come into contact with the temperature measuring unit.

FIG. 10B is a view explaining a principle in which the electrostatic capacitance changes between the conductors, and illustrates a state of the charge between the electrodes when the human body comes into contact with the temperature measuring unit.

FIG. 11 is a flowchart of body temperature measurement of the electronic thermometer.

FIG. 12 is a schematic diagram of an electronic thermometer according to a first modification of the first embodiment.

FIG. 13A is a schematic diagram of an electronic thermometer according to a second modification of the first embodiment, and illustrates a section of a probe unit.

FIG. 13B is a schematic diagram of the electronic thermometer of the second modification, and illustrates a section of a conductor.

FIG. 13C is a schematic diagram of the electronic thermometer of the second modification, and a partially broken perspective view illustrating the probe unit.

FIG. 14 is a schematic sectional view of an electronic thermometer according to a third modification of the first embodiment.

FIG. 15 is a perspective view illustrating a state of a portion in which an electrode and an inner case are fixed to each other in a second embodiment of the invention.

FIG. 16 is a perspective view of the electrode of the second embodiment of the invention.

FIG. 17 is a perspective view of part (electrode fixing unit) of the inner case of the second embodiment of the invention.

FIG. 18 is a perspective view illustrating a state of a portion in which an electrode and an inner case are fixed to each other in a third embodiment of the invention.

FIG. 19 is a perspective view of the electrode of the third embodiment of the invention.

FIG. 20 is a perspective view of part (electrode fixing unit) of the inner case of the third embodiment of the invention.

FIG. 21A is a schematic diagram explaining a configuration of an electronic thermometer 1c according to a fourth embodiment of the invention, and a perspective view of part (electrode fixing unit) of an electrode and an inner case.

FIG. 21B is a schematic diagram explaining the configuration of the electronic thermometer 1c of the fourth embodiment of the invention, and a sectional view of the part (electrode fixing unit) of the electrode and the inner case.

### BEST MODES FOR CARRYING OUT THE INVENTION

An exemplary embodiment of the invention will be described below with reference to the drawings. However, unless otherwise noted, a scope of the invention is not limited to a size, a material, and a shape of a component described in the embodiment and a relative disposition of the components.

### First Embodiment

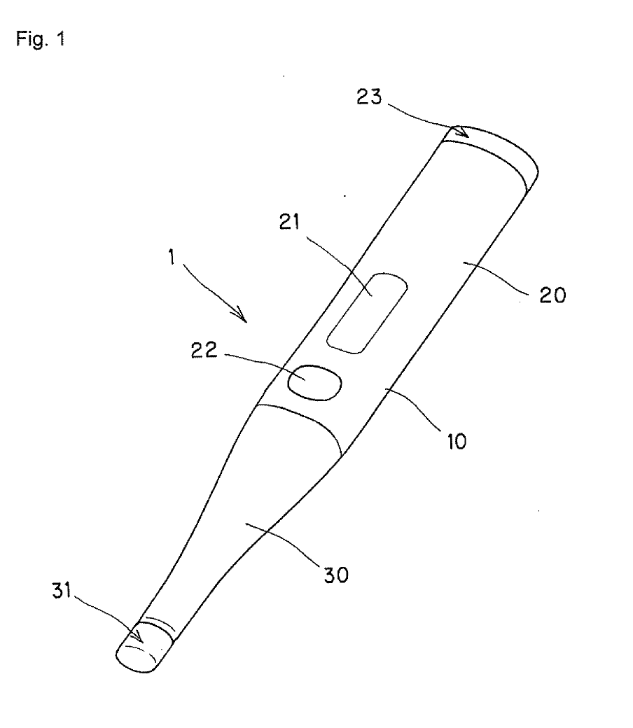

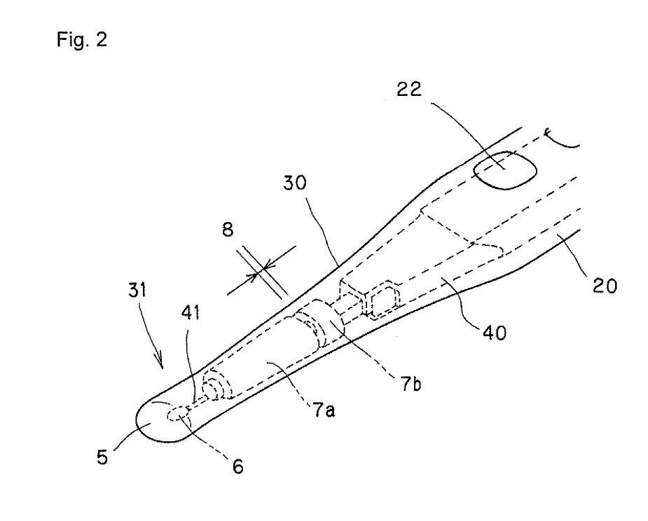

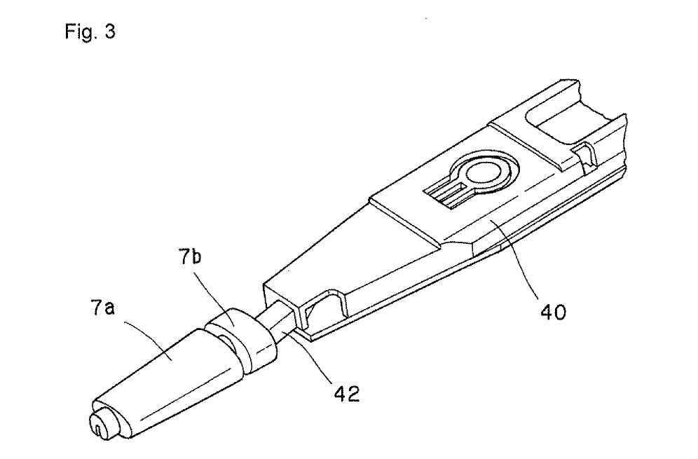

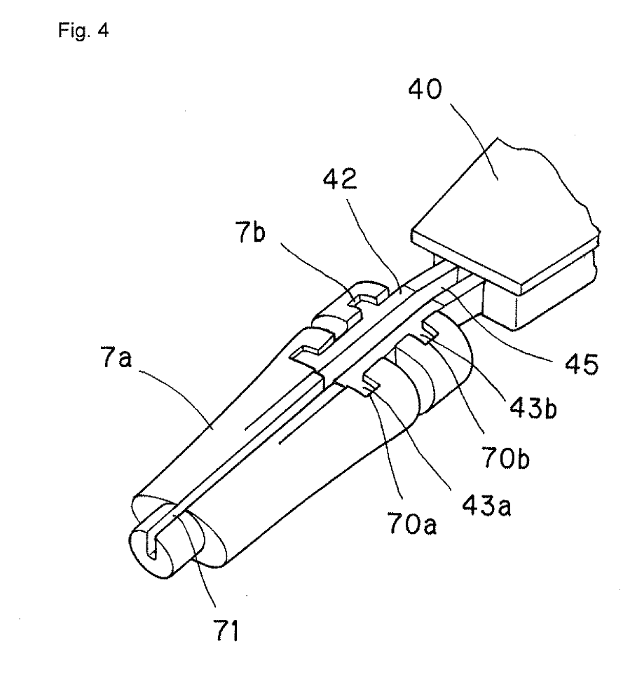

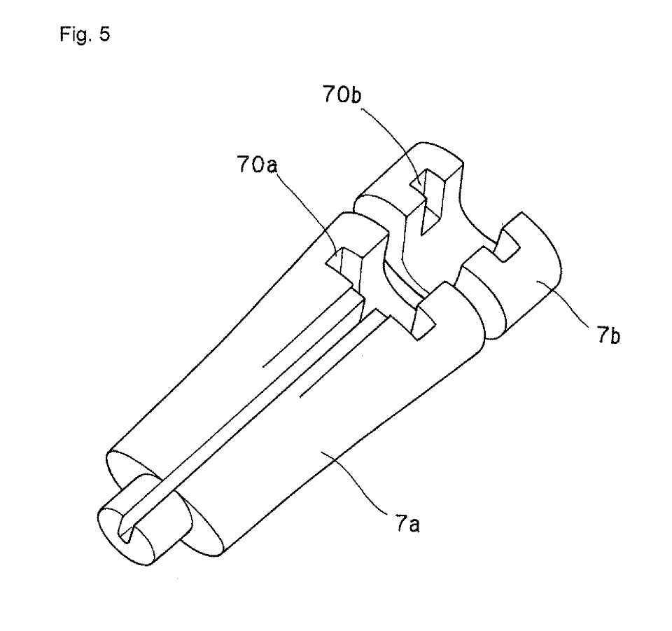

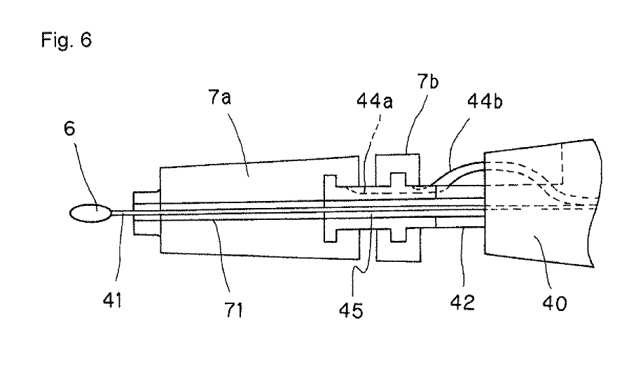

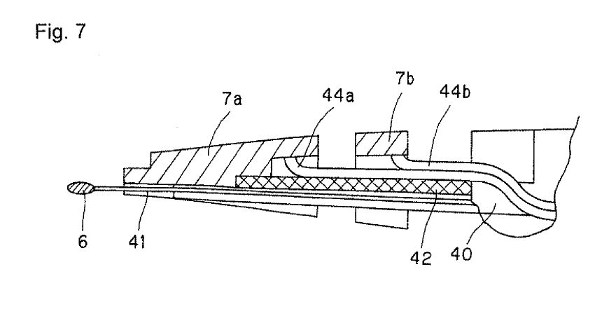

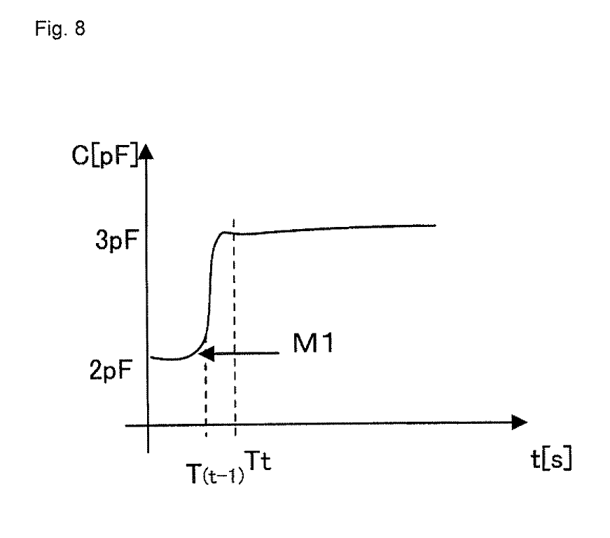

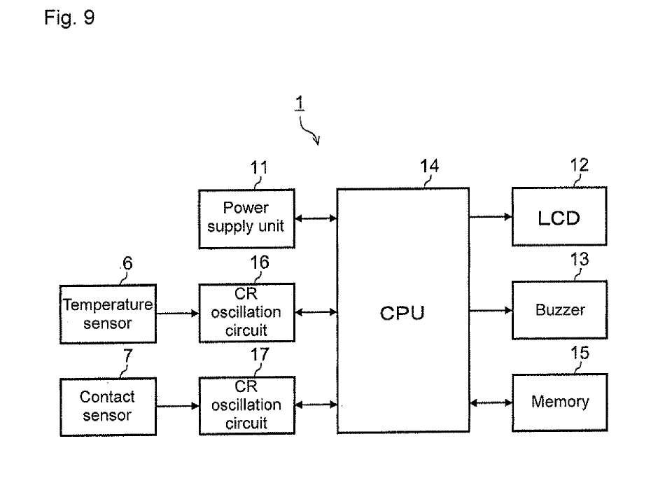

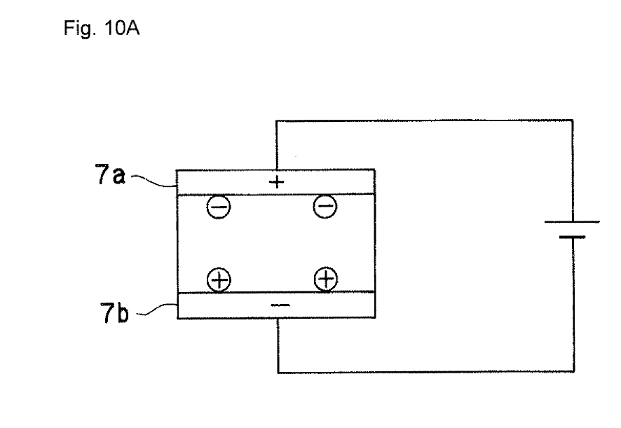

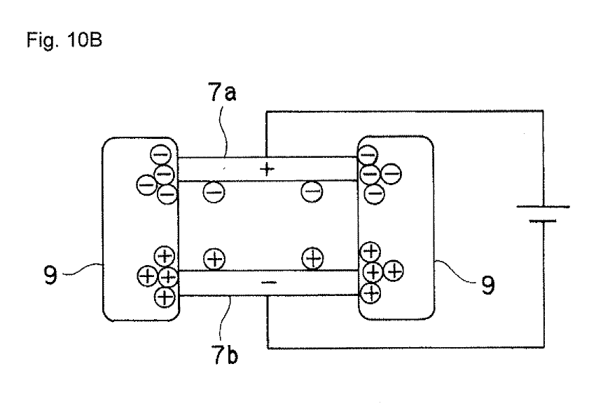

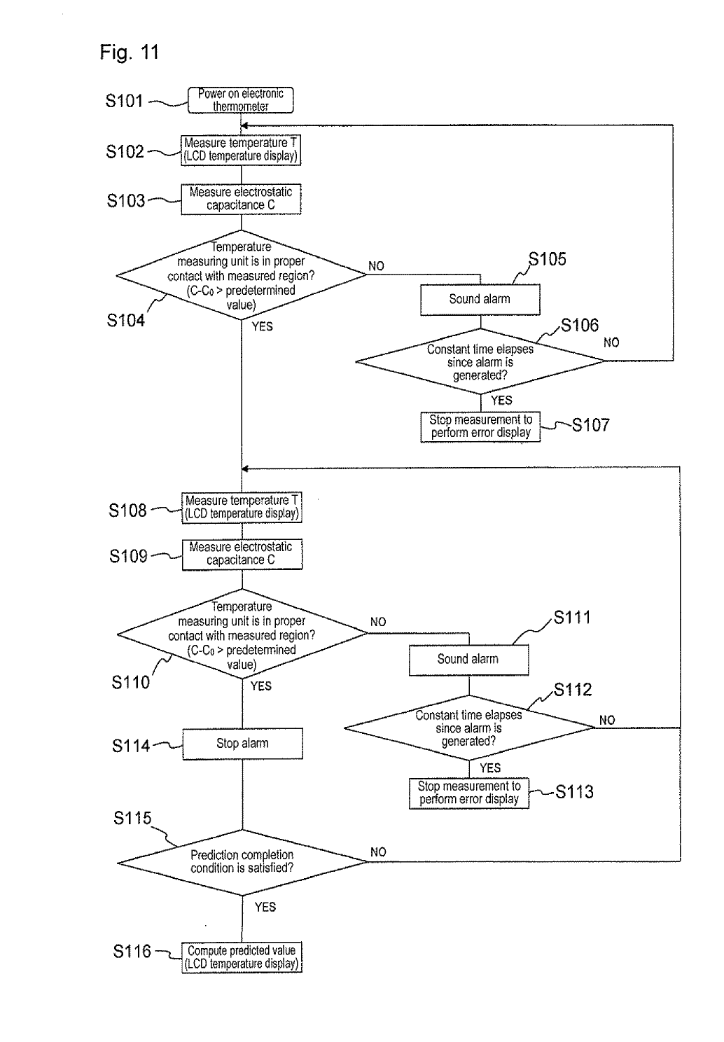

An electronic thermometer according to a first embodiment of the invention will be described with reference to FIGS. 1 to 11. FIG. 1 is a perspective view of the electronic thermometer of the first embodiment of the invention. FIG. 2 is an enlarged perspective view illustrating a periphery of a probe unit of the electronic thermometer of the first embodiment of the invention. FIG. 3 is an enlarged perspective view illustrating part of an inner case of the first embodiment of the invention. FIG. 4 is a perspective view illustrating the other side of FIG. 3. FIG. 5 is a perspective view of an electrode of the first embodiment of the invention. FIG. 6 is a plan view explaining a wiring configuration of the electronic thermometer of the first embodiment of the invention. FIG. 7 is a sectional view explaining the wiring configuration of the electronic thermometer of the first embodiment of the invention. FIG. 8 is a graph illustrating a state of a change in electrostatic capacitance when a measured region comes into proper contact with a temperature measuring unit, and a horizontal axis indicates a time (s) and a vertical axis indicates the electrostatic capacitance (pF) in the graph. FIG. 9 is a schematic block diagram illustrating an electric configuration of the electronic thermometer. FIG. 10 is a view explaining a principle in which the electrostatic capacitance changes between conductors by contact with a human body, FIG. 10A illustrates a state of a charge between electrodes when the human body does not come into contact with the temperature measuring unit, and FIG. 10B illustrates a state of the charge between the electrodes when the human body comes into contact with the temperature measuring unit. FIG. 11 is a flowchart of body temperature measurement of the electronic thermometer of the first embodiment.

<Outline of Electronic Thermometer>

An outline of the electronic thermometer of the first embodiment will be described with reference to FIGS. 1 to 3.

As illustrated in FIG. 1, an electronic thermometer 1 of the first embodiment of the invention includes a hollow outer case (chassis) 10 that has a water-resistant property while constituting an appearance. The outer case 10 includes a main body portion 20 and a probe unit 30. The main body portion 20 includes a display unit 21, a switch 22, and a battery cover 23 that is used to exchange a power supply such as a battery. A temperature measuring unit 31 is provided at a leading end of the probe unit 30 to abut on a measured region such as an underarm and a sublingual region. For example, the outer case 10 is made of an ABS resin or an elastomer.

As illustrated in FIG. 2, in the electronic thermometer 1, various main inside components (such as a circuit board, a power supply, a display panel such as an LCD, and a buzzer) are attached on the inner case 40. The inner case 40 on which various inside components are attached is mounted on the outer case 10.

The temperature measuring unit 31 provided at the leading end of the probe unit 30 includes a cap 5 that is made of stainless steel (SUS) or the like and a temperature sensor 6, such as a thermistor, which is embedded in and fixed to the inside of the cap 5 by a bonding agent. The temperature sensor 6 is electrically connected to a CR oscillation circuit of the inner case 40 through a lead wire 41 that extends through the hollow center of the probe unit 30 from the inner case 40. The temperature sensor 6 changes a resistance value according to heat transferred from an outer surface of the temperature measuring unit 31 (cap 5). The change in the resistance value is output to the CR oscillation circuit to perform body temperature measurement.

As illustrated in FIG. 2, in the electronic thermometer 1 of the first embodiment, a pair of conductors 7a and 7b is disposed as a contact sensor in the hollow center of the probe unit 30.

<Contact Sensor>

A configuration of the contact sensor in the electronic thermometer 1 will be described with reference to FIGS. 2 to 7.

As illustrated in FIG. 2, the pair of conductors 7a and 7b is made of a material such as aluminum, phosphor bronze, copper, and SUS, and are disposed adjacent to each other in a longitudinal direction in the hollow center of the probe unit 30 while separated from each other with a predetermined interval (gap 8). Outer circumferential surfaces of the conductors 7a and 7b are configured to come into tight contact with an inner surface of the probe unit 30 such that an air dielectric layer is not formed between the conductors 7a and 7b and the human body.

As illustrated in FIGS. 3 to 5, the pair of conductors 7a and 7b is fixed to an electrode fixing unit 42 that extends from the inner case 40 toward the leading end (temperature measuring unit 31) side of the probe unit 30. Projections 43a and 43b are provided in the electrode fixing unit 42. Recesses 70a and 70b corresponding to the projections 43a and 43b are provided in the conductors 7a and 7b, respectively. The projections 43a and 43b are fitted in the recesses 70a and 70b to fix the conductors 7a and 7b to the electrode fixing unit 42. A groove portion 71 and a groove portion 45 are provided in the conductor 7a and the electrode fixing unit 42, respectively, in order to pass the lead wire 41 that connects the temperature sensor 6 and the inner case 4.

As illustrated in FIGS. 6 and 7, the pair of conductors 7a and 7b fixed to the electrode fixing unit 42 is connected to a circuit board of the inner case 4 through lead wires 44a and 44b while insulated from each other. When a voltage is applied to the pair of conductors 7a and 7b, a charge is accumulated in the pair of conductors 7a and 7b, thereby constituting a pair of electrodes (capacitor). An electrostatic capacitance generated between the conductors (electrode) 7a and 7b changes depending on a difference in permittivity between the air and the human body when the human body comes into contact with outsides of the conductors 7a and 7b with the probe unit 30 interposed therebetween. Therefore, the pair of conductors (electrode) 7a and 7b acts as the contact sensor 7 that senses whether the human body is in contact with the probe unit 30.

The body temperature measurement is performed in the state in which the temperature measuring unit 31 abuts on the measured region while the probe unit 30 is sandwiched between parts of the human body such as the underarm. Accordingly, the contact sensor 7 disposed inside the probe unit 30 can sense the contact state of the human body to detect whether the temperature measuring unit 31 is in proper contact with the measured region.

As illustrated in FIG. 8, the electrostatic capacitance between the conductors 7a and 7b is about 2 pF before the measured region comes into contact with the temperature measuring unit 31, while the electrostatic capacitance becomes about 3 pF after the contact. That is, it is found that the electrostatic capacitance of the contact sensor 7 increases by about 1 pF by the contact of the measured region with the temperature measuring unit 31. In this figure, the numeral M1 designates a moment in which the probe unit is firmly sandwiched in the underarm. Accordingly, for example, a determination whether the temperature measuring unit 31 is in proper contact with the measured region can be made based on the case where the increased amount of the electrostatic capacitance exceeds 0.5 pF.

The increased amount of the electrostatic capacitance increases as a place with which the human body is into contact comes close to the gap. The gap formed between surfaces opposite each other is the shortest distance between the conductors 7a and 7b. In the present embodiment, substantially ring end faces that are opposite each other in an axis direction of the conductors 7a and 7b constitute the surfaces opposite each other. Therefore, the increased amount of the electrostatic capacitance becomes the maximum when the human body comes into contact with a whole circumference of the outer surface of the probe unit 30 along the gap 8 formed between the surfaces opposite each other. At this point, the electrostatic capacitance is set to an electrostatic capacitance in the state in which the temperature measuring unit 31 is in proper contact with the measured region, thereby being able to make the determination whether the temperature measuring unit 31 located at the leading end of the probe unit 30 is firmly sandwiched in the underarm or the like.

The increased amount of the electrostatic capacitance increases with increasing contact area between the probe unit 30 and the human body. Accordingly, a reference increased amount, used to make a determination that the temperature measuring unit 31 is in proper contact with the measured region, is set larger than an increased amount obtained in the state in which the probe unit 30 is held between fingers, which allows a false determination to be prevented.

<Electric Configuration of Electronic Thermometer>

Referring to FIG. 9, the electronic thermometer 1 mainly includes the temperature sensor 6, the contact sensor 7, a power supply unit 11, an LCD 12, a buzzer 13, a CPU (Central Processing Unit) 14, a memory 15, and CR oscillation circuits 16 and 17.

The power supply unit 11 includes a power supply such as a battery to supply an electric power to the CPU 14. The LCD 12 that is of the display unit displays measurement result under the control of the CPU 14. The buzzer 13 that is of informing means for a user sounds an alarm under the control of the CPU 14. The memory 15 that includes a storage device such as a ROM and a RAM is connected to the CPU 14.

The CR oscillation circuit 16 converts the change in the resistance value output from the temperature sensor 6 into a frequency and outputs the frequency to the CPU 14. The CR oscillation circuit 17 converts the change in electrostatic capacitance output from the contact sensor 7 into a frequency and inputs the frequency to the CPU 14.

A principle in which the electrostatic capacitance changes between the conductors (electrode) 7a and 7b will be described with reference to FIGS. 10A and 10B. Although FIGS. 10A and 10B conceptually illustrate the direct contact between the human body 9 and the conductor 7, actually the probe unit 30 is interposed between the human body 9 and the conductor 7.

Because the human body is larger than the air in specific permittivity, a larger amount of charges are generated in the area near the electrode in the human body 9 compared with the air, when the human body 9 comes into contact with the probe unit 30. Therefore, the electrostatic capacitance increases between the conductors 7a and 7b.

The CPU 14 measures the change in electrostatic capacitance to which the frequency-conversion is performed by the CR oscillation circuit 17, and determines whether the temperature measuring unit 31 is in proper contact with the measured region. That is, in the electronic thermometer 1 of the present embodiment, the CPU 14 acts as both the measurement unit and the determination unit of the invention.

<Body Temperature Measurement Flow>

A flow of the body temperature measurement performed by the electronic thermometer 1 of the present embodiment will be described with reference to FIG. 11. At this point, the electronic thermometer 1 of the present embodiment is a prediction type electronic thermometer by way of example.

When the electronic thermometer 1 of the present embodiment is powered on (S101), the CPU 14 starts the temperature detection with the temperature sensor 6 (S102), and starts the electrostatic capacitance detection with the contact sensor 7 (S103). An electrostatic capacitance value C0 (pF) that is detected immediately after the electronic thermometer 1 is powered on is stored in the memory 15. The CPU 14 determines whether the temperature measuring unit 31 comes into proper contact with the measured region based on whether an electrostatic capacitance value C (pF) detected later increases with respect to the electrostatic capacitance value C0 while exceeding a predetermined value (S104). The electronic thermometer 1 is not sandwiched in the underarm yet immediately after the electronic thermometer 1 is powered on. Accordingly, because the change is not generated in the detected electrostatic capacitance C, the CPU 14 determines that the temperature measuring unit 31 is not in proper contact with the measured region (NO in S104), and the buzzer 13 sounds the alarm (S105). The temperature and the electrostatic capacitance are repeatedly detected until the detected electrostatic capacitance value C increases with respect to the electrostatic capacitance value C0, detected immediately after the power-on of the electronic thermometer 1, while exceeding a predetermined value within a predetermined time from the generation of the alarm, that is, until the CPU 14 determines that the temperature measuring unit 31 is in proper contact with the measured region (NO in S104 and NO in S106). The detected value is stored in the memory 15 as needed.

For example, the predetermined value can be set to 0.5 pF. As to examples of the detection conditions, the temperature and the electrostatic capacitance are detected every one second, and the determination whether the temperature measuring unit 31 is in proper contact with the measured region is made in a period of 15 seconds. The conditions are described by way of example, and there is no limitation to the conditions.

When the increased amount (C-C0) of the electrostatic capacitance does not satisfy the predetermined value after a constant time elapses (YES in S106), the CPU 14 determines that the temperature measuring unit 31 is not in proper contact with the measured region, and stops the measurement to display an error on the LCD 12 (S107). On the other hand, when the increased amount (C-C0) of the electrostatic capacitance exceeds the predetermined value within the constant time (YES in S104), the CPU 14 determines that the temperature measuring unit 31 is in proper contact with the measured region, and makes a transition to the body temperature measurement to start prediction measurement (S108).

When the difference (C-C0) between the electrostatic capacitance value detected initially immediately after the start of the prediction measurement and the electrostatic capacitance value detected immediately after the power-on is not lower than a predetermined value (YES in S110), the buzzer 13 stops the alarm (S114), and the CPU 14 continuously detects the electrostatic capacitance with the contact sensor 7 while continuing the temperature measurement until a prediction completion condition is satisfied (NO in S115, S108, and S109). For example, because the temperature measuring unit 31 is deviated, the difference (C-C0) between the detected electrostatic capacitance value and the electrostatic capacitance value detected immediately after the power-on is lower than the predetermined value during the body temperature measurement (NO in S110), the CPU 14 determines that the temperature measuring unit 31 is not in proper contact with the measured region, and the buzzer 13 sounds the alarm (S111). The alarm is continued or repeated until the difference (C-C0) between the detected electrostatic capacitance value and the electrostatic capacitance value detected immediately after the power-on exceeds the predetermined value within a constant time (for example, 15 seconds), that is, until the CPU 14 determines that the temperature measuring unit 31 is in proper contact with the measured region by correcting the deviation of the temperature measuring unit 31 (NO in S110, 5111, and NO in S112).

When the difference (C-C0) between the electrostatic capacitances does not exceed the predetermined value within the constant time since the generation of the alarm while the deviation of the temperature measuring unit 31 is not corrected (YES in S112), the CPU 14 stops the measurement to display the error on the LCD 12 (S113). On the other hand, when the difference (C-C0) between the electrostatic capacitances exceeds the predetermined value within the constant time since the generation of the alarm while the deviation of the temperature measuring unit 31 is corrected (NO in S112 and YES in S110), the buzzer 13 stops the alarm (S114), and the CPU 14 continuously detects the body temperature and the electrostatic capacitance until the prediction completion condition is satisfied (NO in S115).

When the difference (C-C0) between the electrostatic capacitances is maintained at a value larger than the predetermined value while the alarm is not generated (YES in S110), the CPU 14 determines that the proper contact state is maintained, skips the processing in S114, and continuously detects the body temperature and the electrostatic capacitance until the prediction completion condition is satisfied (NO in S115).

When the prediction completion condition is satisfied (YES in S115), the CPU 14 ends the measurement, and computes a predicted value to display the measurement result on the LCD 12 (S116).

### Advantage of First Embodiment

According to the first embodiment, the electrode that detects the contact state with the human body is disposed in the hollow center of the outer case, so that the outer case identical to conventional one can be used. That is, it is not necessary to change the shape of the outer case to a special shape in which the electrode can be disposed. The electrode can be positioned at a proper detection point inside the probe unit by mounting the inner case on the hollow center of the outer case, which facilitates the electrode attaching work.

Accordingly, in the present embodiment, the contact state with the human body can be confirmed by the simple, easy-to-assemble configuration.

<Modifications>

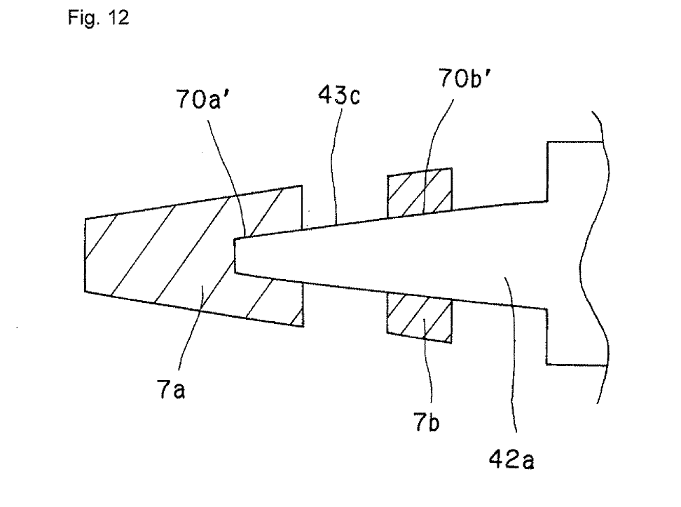

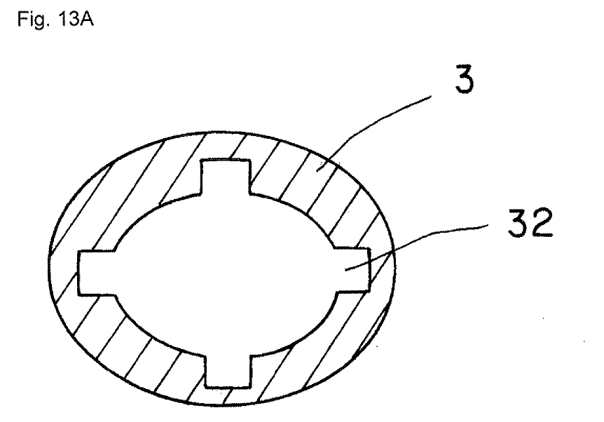

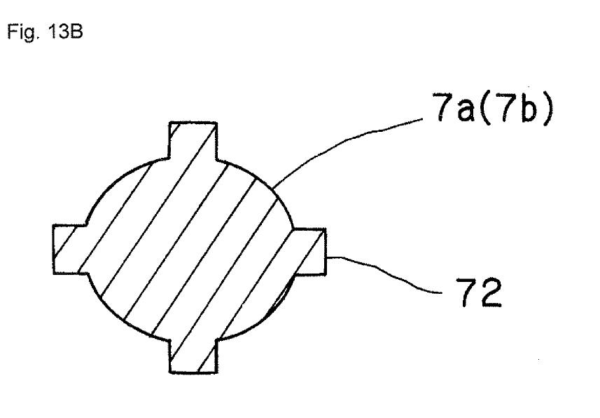

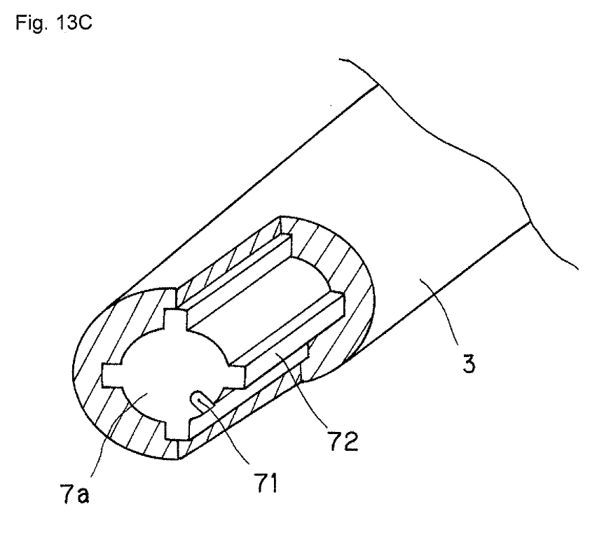

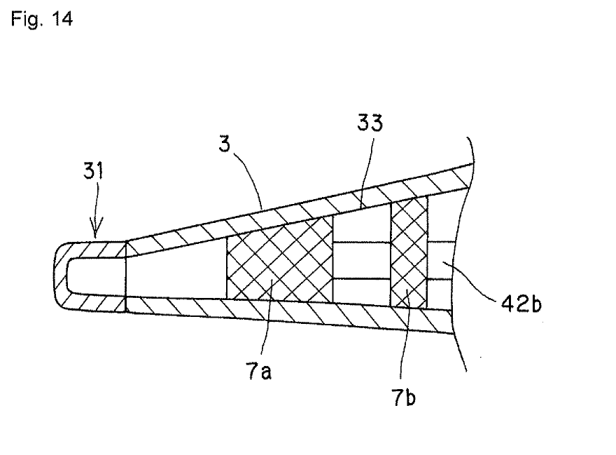

Electronic thermometers according to modifications of the present embodiment will be described with reference to FIGS. 12 to 14. FIG. 12 is a schematic diagram of an electronic thermometer according to a first modification. FIG. 13 is a schematic diagram of an electronic thermometer according to a second modification, and FIG. 13A illustrates a section of a probe unit, FIG. 13B illustrates a section of a conductor, and FIG. 13C is a partially broken perspective view illustrating the probe unit. FIG. 14 is a schematic sectional view of an electronic thermometer according to a third modification.

The method for fixing the pair of conductors and the inner case is not limited to the fitting method of the first embodiment, but various methods may appropriately be adopted. In the electronic thermometer of the first modification illustrated in FIG. 12, instead of the projections 43a and 43b, an inclined or tapered surface 43c is provided in an electrode fixing unit 42a. Inclined or tapered surfaces 70a′ and 70b′ corresponding to the surface 43c are provided in the pair of conductors 7a and 7b. The surface 43c and the surfaces 70a′ and 70b abut on each other to position the pair of conductors 7a and 7b in the electrode fixing unit 42a.

In the electronic thermometer of the second modification illustrated in FIGS. 13A, 13B, and 13C, a groove 32 is provided in an inner wall surface of the probe unit 30 along a direction in which the conductors 7a and 7b fixed to the inner case 4 are inserted in the probe unit 30. A projection (rib) 72 fitted in the groove 32 is provided in an outer circumferential surface of the conductor 7a. Because the contact surfaces of the probe unit 30 and conductors 7a and 7b are formed into the projected and recesses surfaces, a contact area between the probe unit 30 and the conductors 7a and 7b increases to increase the changed amount of the electrostatic capacitance, which allows detection accuracy to be improved. The conductors 7a and 7b is pushed while the projection 72 is fitted in the groove 32, which allows the conductors 7a and 7b to be smoothly inserted (attached). Alternatively, grooves may be provided in the outer circumferential surfaces of the conductors 7a and 7b while projections are provided in the inner wall surface of the probe unit 30.

In the electronic thermometer of the third modification illustrated in FIG. 14, an electrode fixing unit 42b includes an elastic portion, the conductors 7a and 7b are pressed against the inner wall surface 33 of the probe unit 30 by the electrode fixing unit 42b while the inner case is mounted on the outer case, and the conductors 7a and 7b are tightly attached to the inner wall surface 33 of the probe unit 30. Therefore, for example, an air layer except the probe unit 30 is not interposed between the conductors 7a and 7b and the human body, so that the detection accuracy can be improved. When the whole of the electrode fixing unit 42b has the elasticity, possibly the gap between the conductors 7a and 7b is reduced during the assembly depending on a degree in which the electrode fixing unit 42b is pressed. Accordingly, the elastic portion of the electrode fixing unit 42b is preferably set to a portion except the portion between the conductors 7a and 7b. More preferably, in the electrode fixing unit 42b, an elastic member made of an elastomer is integrally molded between a portion, located on the side close to the board, to which the conductor 7b is fixed, and a portion to which the board is fixed in the inner case, and other portions are made of a material identical to that of the inner case. Therefore, with this configuration, the portions to which the conductors 7a and 7b are fixed and the portion between the conductors 7a and 7b can stably be fitted and fixed.

### Second Embodiment

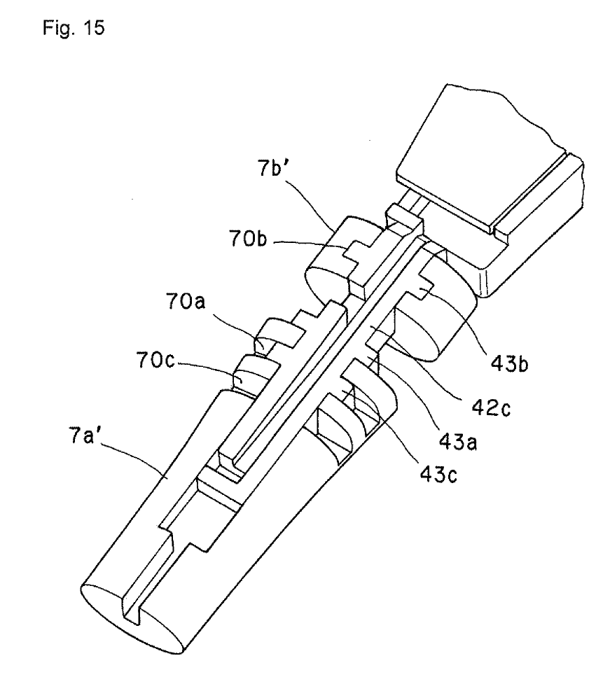

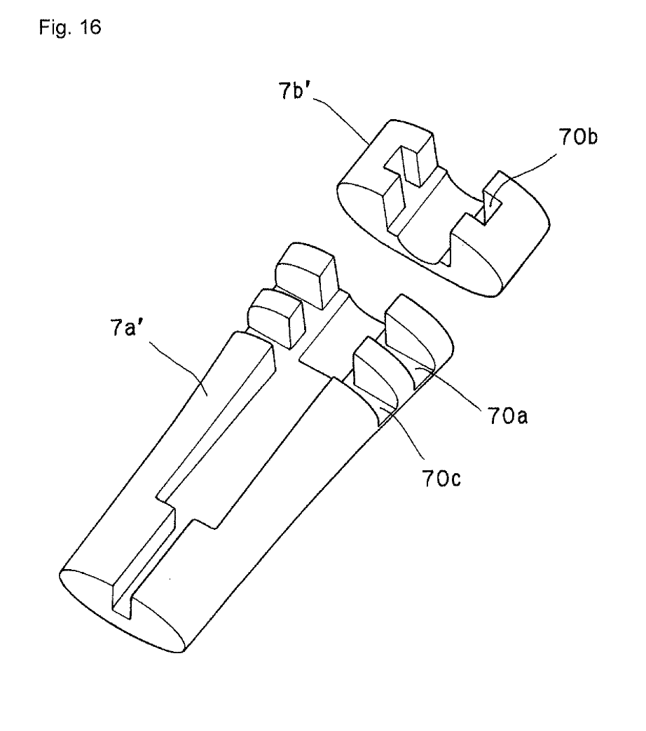

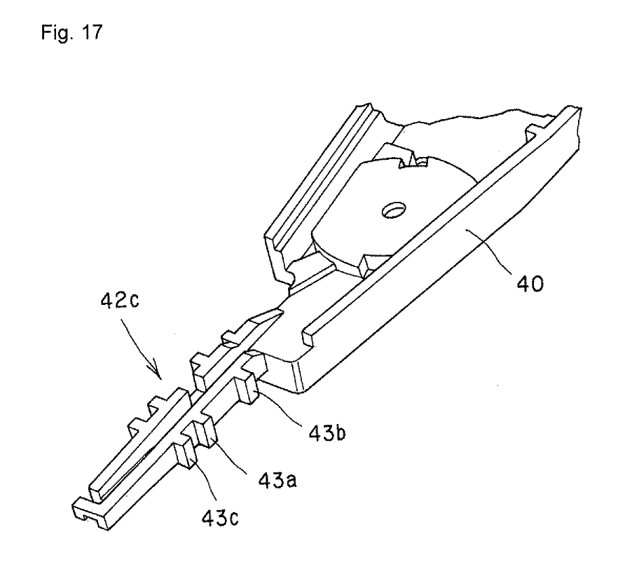

An electronic thermometer 1a according to a second embodiment of the invention will be described below with reference to FIGS. 15 to 17. FIG. 15 is a perspective view illustrating a state of a portion in which an electrode and an inner case (electrode fixing unit) are fixed to each other in the electronic thermometer 1a of the second embodiment of the invention. FIG. 16 is a perspective view of the electrode of the second embodiment of the invention. FIG. 17 is a perspective view of part (electrode fixing unit) of the inner case of the second embodiment of the invention. Only a point different from the first embodiment is described in the second embodiment. The common component and configuration are designated by the similar numerals, and the descriptions are omitted. The action and effect generated by the common component and configuration are also omitted.

In the configuration of the present embodiment, a distance of the gap formed between the pair of conductors can be selected in fixing the conductor to inner case.

As illustrated in FIGS. 15 to 17, two recesses 70a and 70c are provided in a conductor 7a′ and two projections 43a and 43c are provided in an electrode fixing unit 42c. As illustrated in FIG. 15, the gap between the conductor 7a′ and a conductor 7b′ is widened in the state in which the projection 43c is fitted in the recess 70a. On the other hand, although not illustrated, the gap between the conductor 7a′ and the conductor 7b′ is narrowed in the state in which the projection 43a is fitted in the recess 70a while the projection 43c is fitted in the recess 70c.

The gap between the conductors can be adjusted by changing the fitting combination of the recess and the projection. Accordingly, the electronic thermometer is produced while the conductors are fixed with the gap suitable to a body type of the user, so that one kind of the inner case (electrode fixing unit) and one kind of the conductor can meet different product specifications. That is, it is not necessary to prepare multiple kinds of the inner cases (electrode fixing units) and conductors having different attaching positions, and excellent productivity can be obtained.

### Third Embodiment

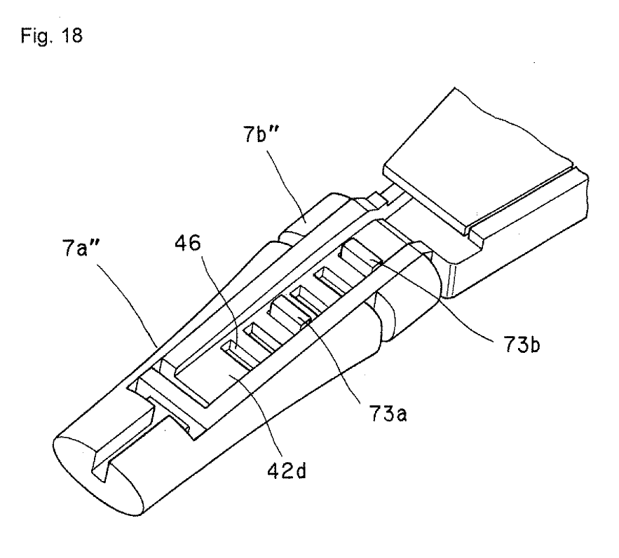

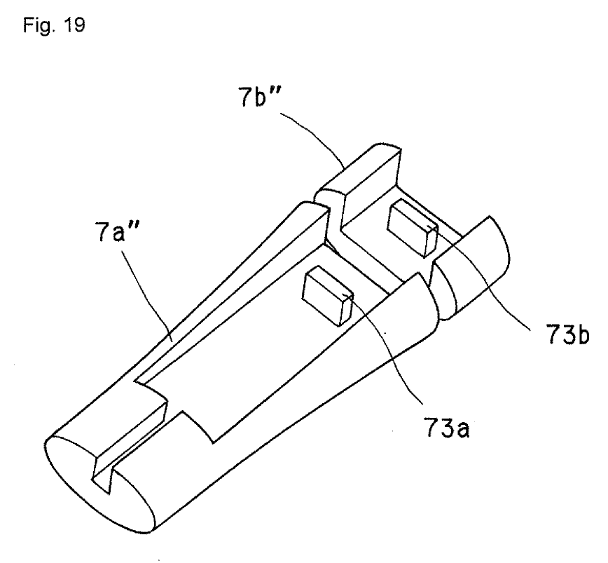

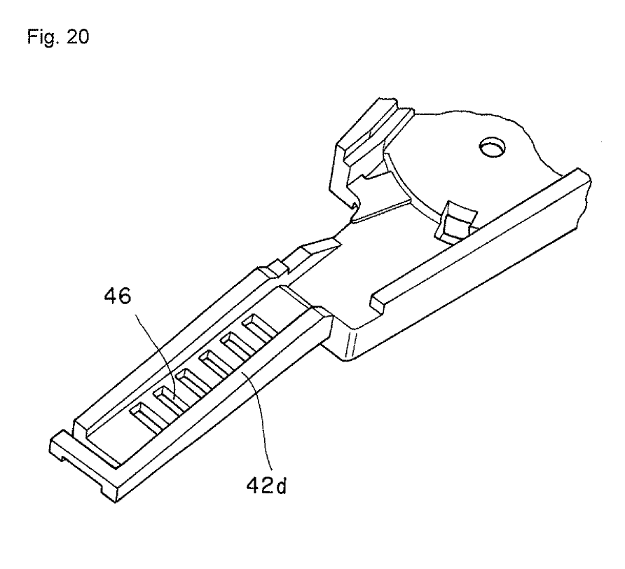

An electronic thermometer 1c according to a third embodiment of the invention will be described below with reference to FIGS. 18 to 20. FIG. 18 is a perspective view illustrating a state of a portion in which an electrode and an inner case (electrode fixing unit) are fixed to each other in the electronic thermometer 1b of the third embodiment of the invention. FIG. 19 is a perspective view of the electrode of the third embodiment of the invention. FIG. 20 is a perspective view of part (electrode fixing unit) of the inner case of the third embodiment of the invention. Only a point different from the embodiments is described in the third embodiment. The common component and configuration are designated by the similar numerals, and the descriptions are omitted. The action and effect generated by the common component and configuration are also omitted.

In the present embodiment, not only the gap between the conductors but also the position where the gap is formed can be selected in fixing the conductor to the inner case.

As illustrated in figures, projections 73a and 73b are provided in conductors 7a″ and 7b″, respectively. A plurality of holes 46 in which the projections 73a and 73b can be fitted are made at equal intervals in an electrode fixing unit 42d along a direction in which the electrode fixing unit 42d extends (the longitudinal direction of the probe unit). The projections 73a and 73b are fitted in the holes 46 to fix the conductors 7a″ and 7b″ to the electrode fixing unit 42d.

The positions where the conductors 7a″ and 7b″ are fixed can be changed by changing the holes 46 in which the projections 73a and 73b are fitted. That is, the gap between the conductors 7a″ and 7b″ can be changed and the gap position can be changed in the probe unit.

Accordingly, the gap suitable to the body type of the user is selected to produce the electronic thermometer, which allows the one kind of the inner case (electrode fixing unit) and the one kind of the conductor to meet different product specifications. That is, it is not necessary to prepare multiple kinds of the inner cases (electrode fixing units) and conductors having different attaching positions, and excellent productivity can be obtained.

### Fourth Embodiment

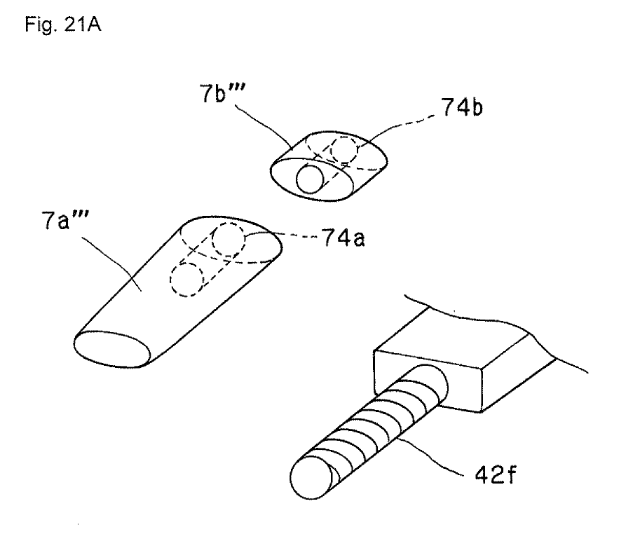

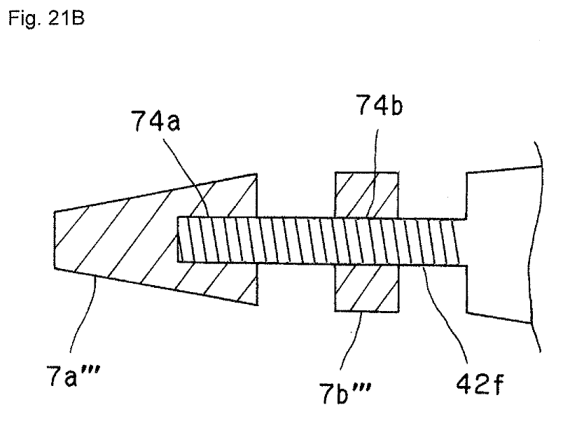

An electronic thermometer 1c according to a fourth embodiment of the invention will be described below with reference to FIG. 21. FIG. 21 is a schematic diagram explaining a configuration of the electronic thermometer 1c of the fourth embodiment of the invention, and FIG. 21A is a perspective view of part (electrode fixing unit) of an electrode and an inner case in the fourth embodiment of the invention, and FIG. 21B is a sectional view of the part (electrode fixing unit) of the electrode and the inner case in the fourth embodiment of the invention.

In the present embodiment, the size of the gap between the conductors and the position where the gap is formed can finely be selected in fixing the conductor to the inner case.

As illustrated in FIGS. 21A and 21B, the portion (electrode fixing unit) in which the pair of conductors is fixed in the inner case constitutes a screw portion 42f in which a male screw is formed in an outer circumferential surface. A non-through screw hole 74a is made in a conductor 7a′″, and a through screw hole 74b is made in a conductor 7b′″. A female screw is formed in an inner circumferential surface of the screw hole 74a, and a female screw is formed in an inner circumferential surface of the screw hole 74b. The screw portion 42f is fitted in the screw holes 74a and 74b to fix the conductors 7a′″ and 7b′″ to the inner case (screw portion 42f) (screw fitting unit). The screw hole 74a of the conductor 7a′″ may be made as the through hole.

The positions where the conductors 7a′″ and 7b′″ are fixed can be changed by changing the position where the screw portion 42f is fitted in the screw holes 74a and 74b. That is, the gap between the conductors 7a′″ and 7b″ can be changed and the gap position can be changed in the probe unit. Particularly, because the position can be changed by adjusting the position where the screw portion is fitted, the position can more finely be changed compared with the third embodiment.

Accordingly, the gap suitable to the body type of the user is selected to produce the electronic thermometer, which allows the one kind of the inner case (electrode fixing unit) and the one kind of the conductor to meet different product specifications. That is, it is not necessary to prepare multiple kinds of the inner cases (electrode fixing units) and conductors having different attaching positions, and excellent productivity can be obtained.

The configurations of the embodiments are described only by way of example. The invention is not limited to the embodiments, but various modifications can be made without departing from the technical thought of the invention. The configurations of the embodiments may be combined.

### DESCRIPTION OF SYMBOLS

1 electronic thermometer
10 outer case
20 main body portion
30 probe unit
31 temperature measuring unit
40 inner case
5 cap
6 temperature sensor
7 contact sensor
7a and 7b conductor (electrode)
8 gap
9 human body
11 power supply unit
12 LCD
13 buzzer
14 CPU
15 memory
16 and 17 CR oscillation circuit

1. An electronic thermometer comprising:
a hollow outer case that includes a probe unit, the probe unit including a temperature measuring unit that abuts on a measured region of a user at a leading end thereof, a temperature sensor being disposed in the temperature measuring unit in order to detect a temperature;
an inner case that is mounted on a hollow center of the outer case while a electronic circuit board is attached to the inner case, a control circuit that processes data detected with the temperature sensor being formed in the electronic circuit board; and
a pair of electrodes that is fixed to the inner case, the electrodes that are not exposed to an outside of the probe unit being positioned inside the probe unit by mounting the inner case on the outer case,
wherein a determination unit is provided in the control circuit, the determination unit measuring an electrostatic capacitance between the pair of electrodes and determining whether the probe unit is in proper contact with the measured region of the user based on a change of the measured electrostatic capacitance.

2. The electronic thermometer according to claim 1, wherein the pair of electrodes is disposed in a longitudinal direction of the probe unit while separated from each other with an interval.

3. The electronic thermometer according to claim 1, wherein the pair of electrodes is fixed to the inner case by fitting a recess or a projection, provided in the pair of electrodes, and a projection or a recess, provided in the inner case, in each other.

4. The electronic thermometer according to claim 3, wherein the pair of electrodes and/or the inner case includes the plurality of recesses or projections.

5. The electronic thermometer according to claim 1, wherein screw fitting units, which are being able to be fitted in each other, are provided in the pair of electrodes and the inner case.

6. The electronic thermometer according to claim 1, wherein an electrode fixing unit in the inner case includes an elastic portion, and the pair of electrodes is positioned by pressing the electrodes against an inner wall surface of the probe unit such that the electrodes are attached firmly to the inner wall surface of the probe unit.

7. The electronic thermometer according to claim 2, wherein the pair of electrodes is fixed to the inner case by fitting a recess or a projection, provided in the pair of electrodes, and a projection or a recess, provided in the inner case, in each other.

8. The electronic thermometer according to claim 2, wherein screw fitting units, which are being able to be fitted in each other, are provided in the pair of electrodes and the inner case.

9. The electronic thermometer according to claim 2, wherein an electrode fixing unit in the inner case includes an elastic portion, and the pair of electrodes is positioned by pressing the electrodes against an inner wall surface of the probe unit such that the electrodes are attached firmly to the inner wall surface of the probe unit.

10. The electronic thermometer according to claim 7, wherein the pair of electrodes and/or the inner case includes the plurality of recesses or projections.
